(19) United States
(12) Patent Application Publication
(10) Pub. No.: US 2023/0415370 A1
(43) Pub. Date: Dec. 28, 2023

(54) SOLUTION FOR ESTIMATING DRYING SHRINKAGE OF VENEER

(71) Applicant: RAUTE OYJ, Nastola (FI)

(72) Inventors: Jyrki PESONEN, Kajaani (FI); Tuomas PUOSKARI, Kajaani (FI)

(21) Appl. No.: 18/035,359

(22) PCT Filed: Nov. 3, 2021
(86) PCT No.: PCT/FI2021/050745

§ 371 Date: May 4, 2023

(30) Foreign Application Priority Data
Nov. 4, 2020 (FI) 20206103

(51) Int. Cl. B27L 5/08; B27L 5/02; G01N 33/46; G01N 9/24

(52) U.S. Cl. CPC B27L 5/08; B27L 5/02; G01N 33/46; G01N 9/24

(57) ABSTRACT

The invention relates to a system for generating an estimation of a drying shrinkage of a veneer sheet, the system comprising: a density measurement device for generating data representing a density of the veneer sheet; at least one other entity suitable to generate data representing at least one characteristic of the veneer sheet an apparatus for receiving data from the density measurement device and data from the at least one other entity, the apparatus arranged to generate an estimation of a drying shrinkage of the veneer sheet based on the received data. The invention also relates to an apparatus, a method, and a computer program product.

### TECHNICAL FIELD

The invention concerns in general the technical field of wood product production. More particularly, the invention concerns veneer production.

### BACKGROUND

Manufacturing of wood products is a process in which a plurality of production related parameters is taken into account in order to achieve an efficient manufacturing process. In a plywood and veneer production a material is manipulated during the manufacturing process in different ways to optimize a final product, i.e. a plywood. The manipulation technique is a process phase dependent and an effect of the manipulation in a certain phase needs to be taken into consideration in one or more latter phases. For example, the raw material in the plywood and veneer production is typically wood log which is conditioned for the latter phases of the production. The conditioning refers to heating and moisturizing the logs so that the wood material is optimal for a peeling device arranged to generate thin continuous sheet of veneer i.e. veneer ribbon. In other words, the raw material is moisturized to optimize the yield and quality in the peeling. However, the latter phases of the plywood and veneer production require that the veneer ribbon is cut to discrete sheets of veneer which are dried at least to some extent to prepare them for gluing together for generating e.g. plywood panels with desired dimensions.

It is commonly known from material sciences that drying of material causes shrinkage of the material in at least most of the cases. This is true at least in wood products and generates impact in the plywood and veneer production in a sense that advantageously the shrinkage is taken into account in cutting to minimize waste in the production. Traditional approach in taking the shrinkage into account is to monitor wood density since according to cumulative experience and research it is detected that the higher the density of the wood the greater the observed volumetric shrinkage. Moreover, it is also known that defects, such as knots or rotting of the wood cause effect on the shrinkage of the wood i.e. the shrinkage of the veneer sheet when drying.

The drying shrinkage is tackled especially in plywood and veneer production from coniferous wood with a model representing veneer sheet shrinkage (e.g. tangentially) with respect to moisture content. However, the model has shown to be unreliable and not optimal for plywood and veneer production. Hence, there is need to develop solutions for improving the situation.

### SUMMARY

The following presents a simplified summary in order to provide basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

An object of the invention is to present a system, an apparatus, a method, and a computer program product for generating an estimation of a drying shrinkage of a veneer sheet.

The objects of the invention are reached by a system, an apparatus, a method, and a computer program product as defined by the respective independent claims.

According to a first aspect, a system for generating an estimation of a drying shrinkage of a veneer sheet is provided, the system comprising: a density measurement device for generating data representing a density of the veneer sheet; at least one other entity suitable to generate data representing at least one characteristic of the veneer sheet; an apparatus for receiving data from the density measurement device and data from the at least one other entity, the apparatus arranged to generate an estimation of a drying shrinkage of the veneer sheet based on the received data.

The other entity may be at least one of the following: a peeling device; an image capturing device; a moisture measurement device; data storage storing data representing characteristics of raw material; data storage storing data representing expected characteristic after a predefined phase of a manufacturing process.

For example, the apparatus of the system may be arranged to apply a classification function to the data representing at least one characteristic of the veneer sheet for classifying the veneer sheet to a class among a plurality of classes in accordance with the data representing at least one characteristic of the veneer sheet. The apparatus of the system may also be arranged to access to one or more internal parameters defined individually for each of the classes, wherein the one or more internal parameters are combined with the data received from the density measurement device for generating the estimation of the drying shrinkage of the veneer sheet. Moreover, the apparatus of the system may be arranged to combine the one or more internal parameters and the data received from the density measurement device by using internal parameters of a class as constants of formulas and using respective values representing density characteristics of the veneer sheet as parameters of respective formulas.

Still further, a measurement with one of: the density measurement device; the image capturing device; the moisture measurement device may be arranged to be performed to a veneer ribbon prior to a cutting of the veneer sheets from the veneer ribbon. The measurement is arranged to be performed to a veneer ribbon by defining, by the apparatus of the system, at least one veneer sheet generatable from the veneer ribbon and generating a plurality of measurement values for the at least one veneer sheet with at least one of: the density measurement device; the image capturing device; the moisture measurement device.

The apparatus of the system may be configured to select an estimation model among a plurality of estimation models for estimating the drying shrinkage in accordance with a value representing of the moisture of the veneer sheet.

The apparatus of the system may be configured to perform the generation of the estimation of the drying shrinkage of the veneer sheet by scaling a value representing a preliminary drying shrinkage with a value derivable from at least one measurement value representing moisture.

The apparatus of the system may be arranged to generate a control signal to a cutting device in accordance with the estimation of the drying shrinkage of the veneer sheet to cause a cutting of the veneer sheet from the veneer ribbon.

According to a second aspects, an apparatus for generating an estimation of a drying shrinkage of a veneer sheet is provided, the apparatus comprising: at least one processor; and at least one memory including computer program code; the at least one memory and the computer program code configured to, with the at least one processor, cause the apparatus to: receive data from a density measurement device, the data received from the density measurement device representing a density of the veneer sheet; and receive data from at least one other entity, the data received from the at least one other entity representing at least one characteristic of the veneer sheet; generate an estimation of a drying shrinkage of the veneer sheet based on the received data.

The apparatus may be caused to apply a classification function to the data representing at least one characteristic of the veneer sheet for classifying the veneer sheet to a class among a plurality of classes in accordance with the data representing at least one characteristic of the veneer sheet. The apparatus may be caused to access to one or more internal parameters defined individually for each of the classes, wherein the one or more internal parameters are combined with the data received from the density measurement device for generating the estimation of the drying shrinkage of the veneer sheet. The apparatus may be caused to combine the one or more internal parameters and the data received from the density measurement device by using internal parameters of a class as constants of formulas and using respective values representing density characteristics of the veneer sheet as parameters of respective formulas.

The apparatus may be caused to select an estimation model among a plurality of estimation models for estimating the drying shrinkage in accordance with a value representing of the moisture of the veneer sheet.

Moreover, the apparatus may be caused to perform the generation of the estimation of the drying shrinkage of the veneer sheet by scaling a value representing a preliminary drying shrinkage with a value derivable from at least one measurement value representing moisture.

Still further, the apparatus may be further caused to generate a control signal to a cutting device in accordance with the estimation of the drying shrinkage of the veneer sheet to cause a cutting of the veneer sheet from the veneer ribbon.

According to a third aspect, a method for generating an estimation of a drying shrinkage of a veneer sheet is provided, the method, performed by an apparatus, comprises: receiving data from a density measurement device, the data received from the density measurement device representing a density of the veneer sheet; and receiving data from at least one other entity, the data received from the at least one other entity representing at least one characteristic of the veneer sheet; generating an estimation of a drying shrinkage of the veneer sheet based on the received data.

Moreover, the method may comprise applying a classification function to the data representing at least one characteristic of the veneer sheet for classifying the veneer sheet to a class among a plurality of classes in accordance with the data representing at least one characteristic of the veneer sheet.

In the method, one or more internal parameters defined individually for each of the classes may also be accessed to, wherein the one or more internal parameters are combined with the data received from the density measurement device for generating the estimation of the drying shrinkage of the veneer sheet. For example, the one or more internal parameters and the data received from the density measurement device may be combined, in the method, by using internal parameters of a class as constants of formulas and using respective values representing density characteristics of the veneer sheet as parameters of respective formulas.

Also, an estimation model for estimating the drying shrinkage may be selected among a plurality of estimation models in accordance with a value representing of the moisture of the veneer sheet.

Alternatively or in addition, the generation of the estimation of the drying shrinkage of the veneer sheet may be performed by scaling a value representing a preliminary drying shrinkage with a value derivable from at least one measurement value representing moisture.

The method may further comprise: generating a control signal to a cutting device in accordance with the estimation of the drying shrinkage of the veneer sheet to cause a cutting of the veneer sheet from the veneer ribbon.

According to a fourth aspect, a computer program product for generating an estimation of a drying shrinkage of a veneer sheet is provided, which computer program product, when executed by at least one processor, cause an apparatus to perform the method according to the third aspect as defined in the foregoing description.

The expression “a number of” refers herein to any positive integer starting from one, e.g. to one, two, or three.

The expression “a plurality of” refers herein to any positive integer starting from two, e.g. to two, three, or four.

Various exemplifying and non-limiting embodiments of the invention both as to constructions and to methods of operation, together with additional objects and advantages thereof, will be best understood from the following description of specific exemplifying and non-limiting embodiments when read in connection with the accompanying drawings.

The verbs “to comprise” and “to include” are used in this document as open limitations that neither exclude nor require the existence of unrecited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

The embodiments of the invention are illustrated by way of example, and not by way of limitation, in the figures of the accompanying drawings.

FIG. 1 illustrates schematically a veneer production line according to an example embodiment.

FIG. 2 illustrates schematically an example of an implementation according to an example embodiment.

FIG. 3 illustrates schematically another example of an implementation according to another example embodiment.

FIG. 4 illustrates schematically a further example of an implementation according to a further example embodiment.

FIG. 5 illustrates schematically an apparatus according to an example embodiment.

FIG. 6 illustrates schematically a method according to an example embodiment.

### DESCRIPTION OF THE EXEMPLIFYING EMBODIMENTS

The specific examples provided in the description given below should not be construed as limiting the scope and/or the applicability of the appended claims. Lists and groups of examples provided in the description given below are not exhaustive unless otherwise explicitly stated.

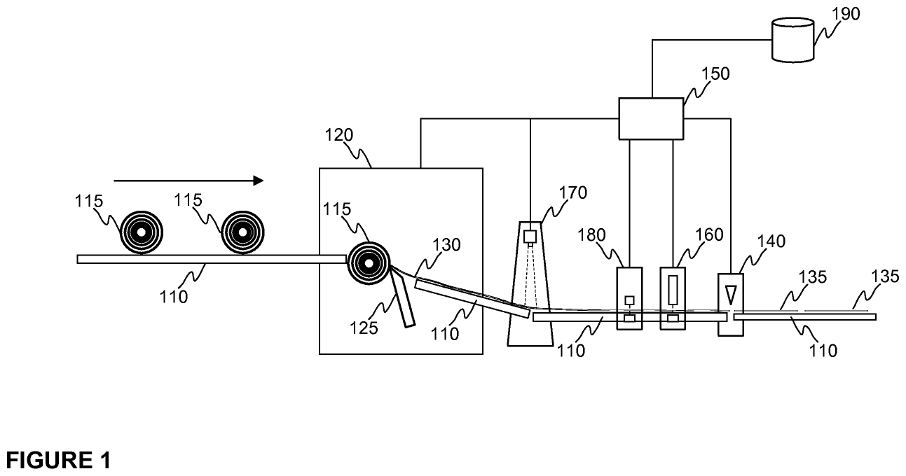

FIG. 1 illustrates schematically a veneer production line, at least in part, into which a solution according to an example embodiment may be implemented to. The selected portions of the veneer production line may comprise a number of conveyor devices 110, such as conveyor belts, by means of which a product may be conveyed along the veneer production line. The number of conveyor devices 110 may vary and may e.g. be selected in accordance of a length of the production line and of a need to separate different production phases with the conveyor devices 110. The conveyor device 110 may be arranged to convey logs 115 to a peeling device 120, i.e. to a lathe, so as to enable peeling of the logs by applying a blade 125 of the peeling device to a log under peeling to generate a continuous veneer sheet 130, i.e. veneer ribbon, as an output of the peeling device 120. Moreover, the veneer production line comprises a cutting machine 140, also called as a clipper, by means of which the veneer ribbon 130 may be cut to discrete veneer sheets 135 with predefined dimensions.

As discussed in the foregoing description the logs are conditioned so as to be optimal for the veneer production. One characteristic is that the logs are moisturized optimally e.g. in accordance with tree species used for the veneer production. As a result the veneer ribbon 130 and the discrete veneer sheets 135 are moist and it may be assumed that in response to drying the dimensions of the discrete veneer sheets 135 will change due to shrinkage. Hence, prior to cutting the veneer ribbon 130 to veneer sheets 135 it is advantageous to generate information, such as an estimation on an expected shrinkage, in order to take it into account in cutting the discrete veneer sheets 135 and sorting them, and as a result, to allow a production of high quality discrete veneer sheets 135. In the described manner a yield of the raw material is higher because the cutting of the veneer sheets 135 may be optimized.

In accordance with an example embodiment a generation of an estimation of the shrinkage of the veneer sheets may be performed with an apparatus 150 arranged to receive data from a plurality of devices, or entities, and to perform an analysis of the received data so as to enable to generate the estimation of the shrinkage of the veneer sheets. The data received by the apparatus 150 comprises at least a number of data values representing a density of the veneer sheet obtained by measuring the veneer ribbon 130 and at least some other data representing at least one characteristic of the veneer sheet or the production line, or its entity, as is described in the forthcoming description. The data representing the density of the veneer sheet may be obtained with a density measurement device 160 which may refer to an apparatus comprising an applicable sensor for obtaining data from the veneer ribbon 130 in accordance with a sampling rate of the density measurement device 160. For example, the density measurement may be based on a use of X-ray equipment, i.e. X-ray tube and a respective sensor, in obtaining data for determining the density. However, other types of devices, such as applying microwaves, may be used for obtaining measurement data to determine the density values. In accordance with an example embodiment the density measurement device 160 may be positioned after the peeling device 120 in the production line e.g. to a measurement position, such as above the veneer ribbon 130. As the veneer ribbon 130 pass by the measurement position of the density measurement device 160 a number of measurement values may be collected from which the density of the veneer ribbon 130 at different positions may be determined.

As mentioned, in accordance with at least some example embodiments the apparatus 150 may receive, in addition to data representing the density of the veneer ribbon, data representing at least one characteristic of the veneer sheet or the production line, or its entity, for estimating a drying shrinkage of the veneer sheet. The information may e.g. be used for controlling a cutting of the veneer ribbon 130 to the discrete veneer sheets 135. The data representing the at least one characteristic of the veneer sheet may e.g. be received from at least one of the following entity: a peeling device 120; an image capturing device 170; a moisture measurement device 180; data storage 190 storing data representing characteristics of a raw material; data storage 190 storing data representing expected characteristic after a predefined phase of a manufacturing process. The data storage may refer to one entity storing all the necessary data or a plurality of data storages each storing a predefined piece of data.

For sake of clarity it is worthwhile to mention that at least some of the characteristics of the veneer sheets may be obtained by measuring the veneer ribbon 130 in the production line before the veneer ribbon 130 is cut to discrete veneer sheets 135. Moreover, some characteristics, such as the ones related to raw material, may be the same for all veneer sheets 135 i.e. not specific for each veneer sheet 135.

In the following it is discussed in more detail, in an exemplified manner, about the aspects, or characteristics, which may be received from the density measurement device 160 and the other entities as mentioned. Some of the aspects may require computation by processing the data received from the respective sensors. The computation may be performed by the respective entity or by the apparatus 150, or the computation may be shared between the mentioned entities.

Aspects received from the density measurement device 160, or at least derivable from data received from the density measurement device 160, may e.g. be:
average density of the veneer sheet determined from a number of measurement values obtained e.g. from veneer ribbon,
density maximum and/or minimum,
variance of the density along a width of the veneer sheet and/or along a length of the veneer sheet,
areal density of the material of the veneer sheet,
areal density of the material of the veneer sheet and water absorbed in the veneer sheet.

It is also possible to derive other values from the measurement data obtained with the density measurement device 160. In accordance with an example, the characteristic applicable to represent the density may be a value representing a total mass of the object measured with the density measurement device 160.

The object may refer to the veneer ribbon 130, or a portion of it. The total density in this context shall be understood as a mass comprising the mass of both the material of the veneer ribbon 130 and the water absorbed by the veneer ribbon 130. However, the mass of water may be subtracted from the total mass by estimating the amount of water in the object e.g. based on information obtainable from the moisture measurement device 180.

Another source of data to be taken into account in the estimation of the drying shrinkage may be the peeling device 120. The data obtainable from the peeling device 120 depends on features of the peeling device 120, but at least the following aspects may be derivable from data obtainable from the peeling device 120:
measurement values relating to raw material, i.e. logs, like diameter, weight, shape (e.g. angle of taper), rounding,
target parameters for the veneer sheet to be peeled, like length and thick-ness,
settings of peeling device, such as technical parameters.

Again, the above given list provides some examples of the aspects, and other aspects may be established based on data obtainable from the peeling device 120.

The image capturing device 170 may refer to so-called machine vision device which is arranged to generate data representing visual aspects of the veneer sheets 135 obtainable from the veneer ribbon 130 from a number of images captured by the image capturing device 170. In other words, the image capturing device 170 may be arranged to analyze captured image data and based on an analysis to generate data representing one or more aspects of the veneer sheets obtainable from the veneer ribbon 130. The aspects may e.g. be
Defects, such as knots, breaches, holes, an amount of rottenness, an amount of bark, defects caused during the manufacturing process. The defects may be expressed as:
   a quantity,
   a size,
   a position on the sheet (e.g. as coordinates),
Combination of defects, such as knot lines expressed as a quantity, a size or a position on the sheet.

Naturally, any other aspects may be brought out when derivable from data obtained with the image capturing device 170.

A further device applicable to generate data from which aspects representing the veneer sheet may be a moisture measurement device 180 whose operation may e.g. be based on an application of microwaves in the measurement (such as measuring change in phase or attenuation when the microwave meets the veneer). The moisture measurement device 180 may obtain measurement data based on which information on at least some of the following aspects may be generated:
average moisture of the veneer sheet determined from a number of measurement values obtained e.g. from veneer ribbon,
moisture maximum and/or minimum,
variance of the moisture along a width of the veneer sheet and/or along a length of the veneer sheet,
a number of moisture pockets,
sizes of moisture pockets,
positions of moisture pockets on the sheet (e.g. as coordinates)

It is also possible to derive other values from the measurement data obtained with the moisture measurement device 160.

Still further, further aspects representing the veneer sheet may be obtained from one or more data storages arranged to store data obtainable from other sources. Such data may e.g. relate to raw material brought into the manufacturing process and/or to a product, such as end product or intermediate product, generated in the production line by defining expected characteristic of the product at a predefined phase of a manufacturing process. This kind of aspects may e.g. be:
Raw material related parameters, such as
   identification of tree of the logs,
   cutting area of the trees,
   time of cutting.
Short log related parameters, such as
   density,
   amount of heartwood/sapwood,
   parameters used in conditioning,
   position of the short log in the full log.
Target moisture of the product at a predefined phase of the production, such as after drying it is allowed that the discrete veneer sheet still comprises moisture of X %.

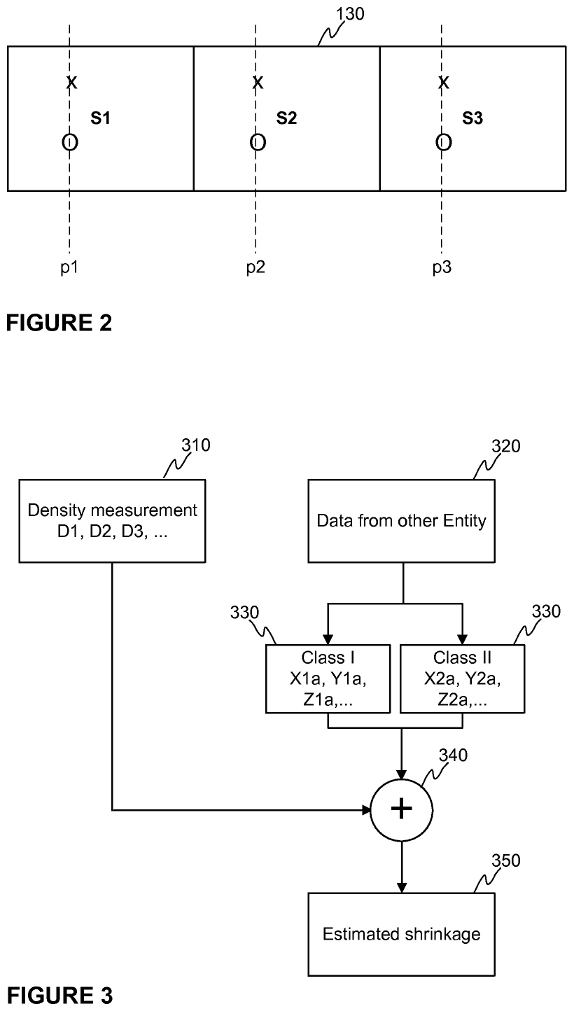

Since the veneer ribbon 130 is lengthy in nature the data obtained with at least the plurality of measurement devices, such as image capturing device 170, a moisture measurement device 180, and the density measurement device 160, is advantageously arranged to represent characteristics of the same portion of the veneer ribbon 130. In other words, since the veneer ribbon 130 is long there may be high variation between different parameters over the length of the veneer ribbon 130 and as a result there may occur variation in a drying shrinkage of the veneer sheets 135 cut from the veneer ribbon 130. This means that in accordance with a desired accuracy of the estimation of the drying shrinkage a sampling rate of the measurement devices may be adjusted to. If the required accuracy is at one veneer sheet 135 level, a measurement may be performed with the respected measurement devices only once over the length of the veneer sheet 135. On the other hand, if an improved accuracy is required, a plurality of measurement shall be performed over the length of the veneer sheet 135. An example of a measurement arrangement may be described by referring to FIG. 2 illustrating, as a non-limiting example, an implementation in which two measurement devices, such as the density measurement device 160 (measurement referred with X) and the moisture measurement device 170 or the image capturing device 180 (measurement referred with O) are arranged to obtain measurement data three times along the length of the veneer ribbon 130. Depending on the measurement arrangement, i.e. if the measurement devices are positioned to obtain the same position of the veneer sheet at the same time or if they are arranged to perform the measurement consecutive to each other, there may be needed a mutual coordination between their operation to take into account the movement of the veneer ribbon 130 over the measurement device in order to control the respective measurement devices to obtain measurement data from the same position of veneer sheets 130 with an acceptable margin.

This may be achieved by detecting a front edge of the veneer sheet and obtaining information on the speed the veneer sheet conveyed in the production line, and especially to the respective measurement devices. By means of this information it is possible to determine an instant of time to perform the measurement so as to receive measurement data being comparable to each other. Reverting back to FIG. 2 the respective devices may be arranged to operate so that they obtain a first measurement data at a position p1 of the veneer ribbon 130, a second measurement data at a position p2 and a third measurement data at a position p3, wherein the positions refer to positions on the veneer ribbon 130, or veneer sheets 135. With this kind of measurement it may be arranged that the veneer ribbon 130 may be divided into sections (referred with S1, S2, and S3 in FIG. 2) advantageously corresponding to the discrete veneer sheets 135 and measurement values may be obtained with respective section as shown. Hence, the accuracy of the data representing characteristics of the veneer sheets 135 may be improved. Naturally, it is still possible to improve the accuracy by increasing the sampling rate of the measurement devices with respect to each section, i.e. by obtaining a plurality of measurement values, such as tens or hundreds, per veneer sheet 135 expressed as sections in FIG. 2. The size of the sections, i.e. the discrete veneer sheets 135, may be defined for the production line as an input parameter.

In the forthcoming description it is described aspects in relation to a generation of an estimation of a drying shrinkage of the veneer sheet 135 according to an example embodiment. Here, it is referred to FIG. 3 schematically illustrating a simplified implementation according to an example embodiment. As mentioned, the density measurement 310 may generate one or more measurement results D1, D2, D3, . . . representing a variety of characteristics, such as an average density (e.g. D1), a maximum density (e.g. D2), and an area density of wood material (e.g. D3), of the veneer sheet in one or more positions of the veneer sheet 135. The mentioned characteristics may be determined on a basis of one or more measurement values. Correspondingly, data representing characteristic of the veneer sheet 135 may be generated 320 by another entity, as described, and the data may represent characteristic of the veneer sheet 135 in one or more positions of the veneer sheet 135, e.g. corresponding to the positions from which the density values are derived. In accordance with an example embodiment the apparatus 150 may be arranged to classify the veneer sheet to a class (cf. Class I, and Class II) 330 among a plurality of predefined classes based on the data values generated 320 by the at least one other entity. Moreover, for each class 330 it is determined internal parameters (referred with X1a, Y1a, Z1a, . . . for Class I and X2a, Y2a, Z2a, . . . for Class II in FIG. 3) which are assigned to the veneer sheet 135 classified to the class in question. The internal parameters may e.g. be defined for each class based on information related to the shrinkage characteristics either directly or indirectly. For example, the parameters may be defined in accordance with tree species used for generating the veneer sheets e.g. by performing preliminary analysis e.g. with a set of samples to understand a behaviour of the veneer sheets when shrunk. In other words, the definition of the parameters may be performed by following a plurality of veneer sheets through the process and by determining the shrinkage per veneer sheet 135 by finding parameters of the veneer sheets 135 having impact to the shrinkage. Such parameters may e.g. be a size of knots and moisture, which are then combined with the data values representing densities. In other words, in response to the classification it is possible to generate 340 an estimation of the drying shrinkage by combining the data values representing densities of the veneer sheet 135 with the internal parameters of the class into which the respective veneer sheet 135 is classified based on data received from the at least one other entity. The generation of the estimation of the drying shrinkage 340 may be performed with a predefined mechanism, such as with a predefined mathematical formula, into which at least some of the values D1, D2, D3, . . . representing characteristics relating to the density and the internal parameters of the respective class (cf. for first class: X1a, Y1a, Z1a, X1b, Y1b, Z1b, X1c, Y1c, Z1c . . . and/or for second class: X2a, Y2a, Z2a, X2b, Y2b, Z2b, X2c, Y2c, Z2c . . . ) may be input. For example, in some implementation the estimation model of the drying shrinkage may be determined with a formula of second degree polynomials:

Drying shrinkage=(X1c*D1²+X1b*D1+X1a)+(Y1c*D2²+Y1b*D2+Y1a)+(Z/c*D3²+Z1b*D3+Z1a) . . .
, wherein D1, D2, D3, . . . correspond to values derivable from a number of density values received from the density measurement device and Xₙₙ, Yₙₙ, Zₙₙ, correspond to internal parameters of the class into which the veneer sheet is classified to. In another implementation the estimation model of the drying shrinkage may be determined with a formula of exponentials:

Drying shrinkage=(X1b*e^{X1a*D1})+(Y1b*e^{Y1a*D2})+(Z1b*^{eZ1a*D3})+ . . .

In some implementations different type of formulas may be combined, for example a polynomial formula might be used with D1 and an exponential formula with D2. Parameters of both formulas may be derived from the class into which the veneer sheet is classified. The formula may e.g. generate a value expressing the drying shrinkage in percent (%). Hence, the estimation of the drying shrinkage may be output 350 from step 340. Generally speaking, the formulas given above are non-limiting examples and other approaches may be applied as well in which the density values are combined with the class parameters in a manner that a value representing the drying shrinkage may be determined. For sake of clarity it is worthwhile to emphasize that the estimation models, i.e. the formulas, provided above are non-limiting examples and they may be adjusted in accordance with a need. For example, the estimation model applied in the calculation of the drying shrinkage may be dependent of a variety of parameters, such as on the value representing the moisture of the veneer sheet in question. For example, it may be assumed that wood shrinkage depends linearly on wood species and change of wood moisture content, if the moisture content is below saturation point of wood fibres. Normally, during veneer peeling, the moisture content of wood is higher than saturation point, but in response to a detection, e.g. based on a number of the measurement values received from the moisture measurement device 180, that the moisture ratio of the veneer sheet is less than a saturation point of the veneer sheet, the formula may be adjusted in order to receive more accurate estimation on the drying shrinkage. In other words, the estimation of the drying shrinkage may be calculated by selecting an estimation model, defined e.g. by a formula, among a plurality of estimation models in accordance with the value representing the moisture of the veneer sheet. Alternatively or in addition, the result of an estimation model may be scaled with a scaling factor calculated based on a value obtainable from the moisture measurement device 180, such as a value derivable from at least one measurement value representing moisture. In some sophisticated solutions, there may be set a target dry moisture content in order to apply a certain model representing the shrinkage i.e. if it is detected that the moisture of the veneer sheet is between the target moisture and the saturated moisture, a certain model is applied to. For example, in one implementation the result of the estimation model of the drying shrinkage, called as a preliminary drying shrinkage, may be corrected by a formula:

Drying shrinkage(corrected)=((M1−2)/32)*Drying shrinkage(preliminary)
, wherein M1 represents an average moisture content of the veneer sheet, 2 is a target dry moisture content of the model and 32 is a saturation point of the wood species in question. In some implementations the constants 2 and 32 or one of them may be replaced with parameters from data storage or parameters from respective class of the veneer sheet. For sake of completeness, it is worthwhile to mention that the moisture value may be derived from a single measurement value, or represents a value calculated, e.g. by using statistical analysis, from a plurality of measurement values.

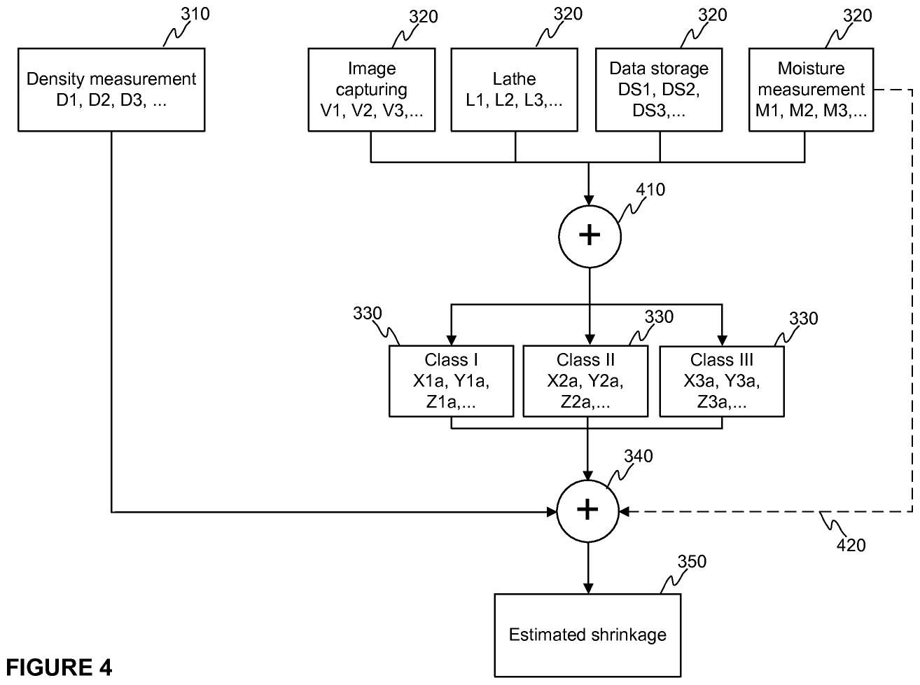

FIG. 4 illustrates schematically further aspects of generating the estimation of the drying shrinkage of the veneer sheet 135 in an example embodiment in which the other data representing characteristics of the veneer sheet 135 is obtained from a plurality of sources. In the non-limiting example, the sources are a moisture measurement device 180 generating measurement values M1, M2, M3, . . . , an image capturing device 170 generating measurement values V1, V2, V3, . . . , a peeling device 120 generating and providing measurement and/or configuration values L1, L2, L3, . . . , and data storage 190 generating and providing values DS1, DS2, DS3, . . . e.g. representing characteristics of the raw material of the veneer sheet 135. The inputs from the plurality of sources 320 may be input to a classification function 410, which is arranged to apply a predefined plan (i.e. a classification model) defined by one or more predefined rules to the inputs and, as a result, to classify the veneer sheet 135 in question to one of the classes (the classes referred with 330 in FIG. 4) in accordance with the input values. As already mentioned in the description of FIG. 3 internal parameters (X1a, Y1a, Z1a, . . . ; X2a, Y2a, Z2a, . . . ; X3a, Y3a, Z3a, . . . ) may be defined for the classes 330, and applied in step 340 with the one or more characteristics representing the density 310 when generating the estimation, or estimations, of the drying shrinkage which is finally output from the process 350.

For sake of completeness the classification function 410 may be arranged to operate so that it monitors the one or more predefined rules, which may e.g. correspond to one or more parameters representing the veneer sheet 135 based on which it may be classified to a certain class. For example, in some implementations the classification function 410 may compare data from plurality of sources 320, one by one, to predefined limits of classes and reduce the number of the classes, until only one class is left. Parameters of that class may be used in step 340.

In some example embodiments, in the generation of the drying estimate in step 340 at least one piece of information relating to moisture may also be taken into account. For example, an average moisture, as referred e.g. with M1, may be input in the formula of generating the estimation of the drying shrinkage, as shown in the foregoing description, in order to improve an accuracy of the estimation. Such an input is referred with reference number 420 in FIG. 4. For sake of clarity it is worthwhile to mention that the input is optional, and it may be valid only with some example implementations of the present invention, such as in the context wherein a preliminary estimation of the drying shrinkage is corrected with a factor being e.g. dependent on the moisture of the veneer sheet as e.g. suggested with a formula in the foregoing description.

In accordance with an embodiment the classification function 410 may be defined manually by an operator, such as a commissioning engineer, of the system based on a large amount of data collected from the system or similar systems. In some examples definition or tuning of the classification function 410 may be arranged as a computer implemented task e.g. by utilizing an artificial intelligence in the task. For example, a neural network may be trained to perform the tuning task by providing input data defining a number of parameters, i.e. characteristics, of the veneer sheets 135, that have been used in classification, and data defining the actual shrinkage of the veneer sheets after drying.

Further in accordance with an embodiment the parameters of the classes 330 may be defined manually by an operator, such as a commissioning engineer, based on a large amount of data collected from the system or similar systems. In some examples tuning of the class 330 parameters is arranged as a computer implemented task e.g. by utilizing an artificial intelligence in the task. In other words, for example a neural network may be trained to perform the tuning task by providing input data defining a number of parameters, i.e. characteristics, of the veneer sheets 135, that have been used in classification, and data defining the actual shrinkage of the veneer sheets after drying.

In general, the tuning, i.e. an adjustment, of the model including the classification function 410 parameters and shrinkage calculation parameters in the classes 330, may advantageously be achieved to operate so that it allows finding such characteristics from the data by means of which, by classifying, a correlation between an actual drying shrinkage and the characteristic representing the density is better than in the whole data set.

For sake of completeness, it is worthwhile to mention, as a non-limiting example, that a mathematical approach of the classification function 410 may be a multi-dimensional array into which the measurement values, and possibly other values, may be input and it may generate a most optimal class among the plurality of classes as an output.

As described in the foregoing description an apparatus 150 may be arranged to determine one or more estimations of the drying shrinkage of the veneer sheet. In accordance with example embodiments the one or more estimations may be used as parameters to control a cutting of the veneer ribbon 130 into discrete veneer sheets 135. For example, the apparatus 150 may be arranged to generate a control signal to a cutting device 140 to perform the cutting so that the discrete veneer sheet 135 is cut longer than the planned size of the end product so as to take into account the shrinkage of the veneer sheet 135 when dried. In some example embodiments where a plurality of estimations are generated and a more sophisticated understanding of a behaviour of the veneer sheet 135 is derived, such as it may be assumed that the shrinkage is not uniform over the length of the veneer sheet 135, the cutting device 140 may be controlled to perform the cutting in accordance with a predetermined shape. For example, the cutting may be performed in a slanted manner with respect to the length of the veneer sheet 135 so as to take into account, or to cancel, the non-uniform shrinkage in the same direction.

According to an example embodiment a solution for generating the estimation and, finally, to generate a control signal to the cutting device 140 may advantageously be performed individually for each veneer sheet 135 i.e. the width of the veneer sheet 135 is determined before the cutting the veneer sheet 135 from the veneer ribbon so that the characteristics of the portion to be cut is taken into account in the described manner in order to estimate the drying shrinkage of the respective portion of the veneer ribbon so as to cut the veneer sheet 135 having the optimal width. In other words, data from all measurement devices is obtained before cutting so that the estimation may be generated, and, hence, taken into account for determining the position of the cutting. Hence, the portion of the veneer ribbon into which the analysis is performed changes continuously resulting a variety in the cutting width of the veneer sheet 135 in accordance with the estimation of the drying shrinkage to the respective portion.

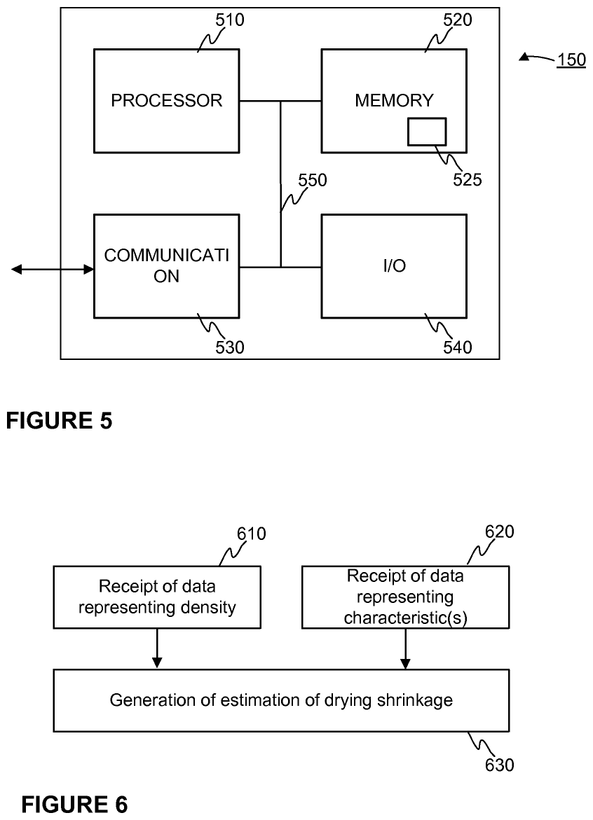

For example, the apparatus 150 may refer to a computing device as schematically illustrated in FIG. 5. FIG. 5 illustrates schematically as a block diagram a non-limiting example of the apparatus 150 applicable to perform the method. The block diagram of FIG. 5 depicts some components of a device that may be employed to implement an operation of the apparatus 150. The apparatus comprises a processor 510 and a memory 520. The memory 520 may store data and computer program code 525. The apparatus may further comprise communication means 530 for wired and/or wireless communication with other entities, such as other systems and/or devices forming the entities as described and similar. Furthermore, I/O (input/output) components 540 may be arranged, together with the processor 510 and a portion of the computer program code 525, to provide a user interface for receiving input from a user and/or providing output to the user of the system when necessary. In particular, the user I/O components may include user input means, such as one or more keys or buttons, a keyboard, a touchscreen or a touchpad, etc. The user I/O components may include output means, such as a display or a touchscreen. The components of the apparatus may be communicatively coupled to each other via a bus 550 that enables transfer of data and control information between the components.

The memory 520 and a portion of the computer program code 525 stored therein may be further arranged, with the processor 510, to cause the apparatus, i.e. the device to perform a method is described in a forthcoming description. The processor 510 may be configured to read from and write to the memory 520. Although the processor 510 is depicted as a respective single component, it may be implemented as respective one or more separate processing components. Similarly, although the memory 520 is depicted as a respective single component, it may be implemented as respective one or more separate components, some or all of which may be integrated/removable and/or may provide permanent/semi-permanent/dynamic/cached storage.

The computer program code 525 may comprise computer-executable instructions that implement functions that correspond to steps of the method when loaded into the processor 510. As an example, the computer program code 525 may include a computer program consisting of one or more sequences of one or more instructions. The processor 510 is able to load and execute the computer program by reading the one or more sequences of one or more instructions included therein from the memory 520. The one or more sequences of one or more instructions may be configured to, when executed by the processor 510, cause the apparatus to perform the method will be described. Hence, the apparatus may comprise at least one processor 510 and at least one memory 520 including the computer program code 525 for one or more programs, the at least one memory 520 and the computer program code 525 configured to, with the at least one processor 510, cause the apparatus to perform the method of generating an estimation of a drying shrinkage of a veneer sheet.

The computer program code 525 may be provided e.g. a computer program product comprising at least one computer-readable non-transitory medium having the computer program code 525 stored thereon, which computer program code 525, when executed by the processor 510 causes the apparatus to perform the method. The computer-readable non-transitory medium may comprise a memory device or a record medium such as a CD-ROM, a DVD, a Blu-ray disc or another article of manufacture that tangibly embodies the computer program. As another example, the computer program may be provided as a signal configured to reliably transfer the computer program.

Still further, the computer program code 525 may comprise a proprietary application, such as computer program code for executing the generation of the estimation of the drying shrinkage of a veneer sheet in the manner as described by the description herein.

Any of the programmed functions mentioned may also be performed in firmware or hardware adapted to or programmed to perform the necessary tasks.

Moreover, as mentioned a functionality of the apparatus may be shared between a plurality of devices as a distributed computing environment. For example, the distributed computing environment may comprise a plurality of devices as schematically illustrated in FIG. 5 arranged to implement the method in cooperation with each other in a predetermined manner. For example, each device may be arranged to perform one or more method steps and in response to a finalization of its dedicated step it may hand a continuation of the process to the next device.

Some aspects of the present invention may relate to a method for generating an estimation of a drying shrinkage of a veneer sheet 135. An example of such a method according to an example embodiment, wherein the method may be performed by an apparatus 150, is schematically illustrated in FIG. 6. The method may comprise: receiving 610 data from a density measurement device 160, the data received from the density measurement device 160 representing a density of the veneer sheet 135. Additionally, the method may comprise: receiving 620 data from at least one other entity, the data received from the at least one other entity representing at least one characteristic of the veneer sheet 135. The steps of receiving the mentioned pieces of data may be performed concurrently or consecutively to each other at least in part. The receipt of data may comprise obtaining it from a respective measurement device or data storage storing the data. In response to the receipt of data the method may comprise a step of generating an estimation of a drying shrinkage of the veneer sheet 135 based on the received data. For example, the generation of the estimation of the drying shrinkage may comprise a sub-step of applying a classification function to the data representing at least one characteristic of the veneer sheet 135 for classifying the veneer sheet 135 to a class among a plurality of classes in accordance with the data representing at least one characteristic of the veneer sheet 135. Further, the apparatus may be caused in the method to access to one or more internal parameters defined individually for each of the classes, wherein the one or more internal parameters are combined with the data received from the density measurement device 160 for generating the estimation of the drying shrinkage of the veneer sheet 135. Still further, the method may comprise that the one or more internal parameters and the data received from the density measurement device 160 are combined by using internal parameters of a class as constants of formulas and using data from density measurement device as parameters of respective formulas. Still further, the method may comprise that results from two or more formulas are combined by summation to achieve more accurate estimation of drying shrinkage of the veneer sheet 135. According to an example embodiment, the method may further comprise: generating a control signal to a cutting device 140 in accordance with the estimation of the drying shrinkage of the veneer sheet 135 to cause a cutting of the veneer sheet 135 from the veneer ribbon 130. Further aspects of the method may have been described in the foregoing description.

The solution described in the foregoing description, and defined in the appended claims, bring a plurality of advantages compared to prior art solutions. Namely, by applying the described solution it is possible to reduce a variation in the width of dried veneer sheets and this allows cutting of the veneer sheets narrower than used to. Additionally, the width of dried veneer sheets may be estimated in an improved way which, in turn, improves a yield of the raw material, because the procedure generates less under wide veneer sheets. Still further, the classification of the veneer sheets in the procedure as described improves a quality of green composed sheets as well as reduces a breakage of the sheets when dried because the sheets are classified in accordance with their characteristics so as to achieve common behaviour during the shrinkage. The drying process may also be adjusted and tailored in accordance with the raw material since the characteristics are known and/or evaluated which improves the outcome of the process. All in all, the product quality e.g. in terms of strength of the product, is achieved at least in part as a result of more accurate drying process.

The specific examples provided in the description given above should not be construed as limiting the applicability and/or the interpretation of the appended claims. Lists and groups of examples provided in the description given above are not exhaustive unless otherwise explicitly stated.

1. A system for generating an estimation of a drying shrinkage of a veneer sheet, the system comprising:
a density measurement device for generating data representing a density of the veneer sheet,
at least one other entity suitable to generate data representing at least one characteristic of the veneer sheet,
an apparatus for receiving data from the density measurement device and data from the at least one other entity, the apparatus arranged to generate an estimation of a drying shrinkage of the veneer sheet based on the received data.

2. The system of claim 1, wherein the other entity is at least one of the following: a peeling device; an image capturing device; a moisture measurement device data storage storing data representing characteristics of raw material; data storage storing data representing expected characteristic after a predefined phase of a manufacturing process.

3. The system of claim 1, wherein the apparatus of the system is arranged to apply a classification function to the data representing at least one characteristic of the veneer sheet for classifying the veneer sheet to a class among a plurality of classes in accordance with the data representing at least one characteristic of the veneer sheet.

4. The system of claim 3, wherein the apparatus of the system is arranged to access to one or more internal parameters defined individually for each of the classes, wherein the one or more internal parameters are combined with the data received from the density measurement device for generating the estimation of the drying shrinkage of the veneer sheet.

5. The system of claim 4, wherein the apparatus of the system is arranged to combine the one or more internal parameters and the data received from the density measurement device by using internal parameters of a class as constants of formulas and using respective values representing density characteristics of the veneer sheet as parameters of respective formulas.

6. The system of claim 1, wherein a measurement with one of: the density measurement device; the image capturing device; the moisture measurement device is arranged to be performed to a veneer ribbon prior to a cutting of the veneer sheets from the veneer ribbon.

7. The system of claim 6, wherein the measurement is arranged to be performed to a veneer ribbon by defining, by the apparatus of the system, at least one veneer sheet generatable from the veneer ribbon and by generating a plurality of measurement values for the at least one veneer sheet with at least one of: the density measurement device; the image capturing device; the moisture measurement device.

8. The system of claim 1, wherein the apparatus of the system is configured to select an estimation model among a plurality of estimation models for estimating the drying shrinkage in accordance with a value representing of the moisture of the veneer sheet.

9. The system of claim 1, wherein the apparatus of the system is configured to perform the generation of the estimation of the drying shrinkage of the veneer sheet by scaling a value representing a preliminary drying shrinkage with a value derivable from at least one measurement value representing moisture.

10. The system of claim 1, wherein the apparatus of the system is arranged to generate a control signal to a cutting device in accordance with the estimation of the drying shrinkage of the veneer sheet to cause a cutting of the veneer sheet from the veneer ribbon.

11. An apparatus for generating an estimation of a drying shrinkage of a veneer sheet, the apparatus comprising:
at least one processor; and
at least one memory including computer program code;
the at least one memory and the computer program code configured to, with the at least one processor, cause the apparatus to:
receive data from a density measurement device the data received from the density measurement device representing a density of the veneer sheet; and
receive data from at least one other entity, the data received from the at least one other entity representing at least one characteristic of the veneer sheet,
generate an estimation of a drying shrinkage of the veneer sheet based on the received data.

12. The apparatus of claim 11, wherein the apparatus is caused to apply a classification function to the data representing at least one characteristic of the veneer sheet for classifying the veneer sheet to a class among a plurality of classes in accordance with the data representing at least one characteristic of the veneer sheet.

13. The apparatus of claim 12, wherein the apparatus is caused to access to one or more internal parameters defined individually for each of the classes, wherein the one or more internal parameters are combined with the data received from the density measurement device for generating the estimation of the drying shrinkage of the veneer sheet.

14. The apparatus of claim 13, wherein the apparatus is caused to combine the one or more internal parameters and the data received from the density measurement device by using internal parameters of a class as constants of formulas and using respective values representing density characteristics of the veneer sheet as parameters of respective formulas.

15. The apparatus of claim 11, wherein the apparatus is caused to select an estimation model among a plurality of estimation models for estimating the drying shrinkage in accordance with a value representing of the moisture of the veneer sheet.

16. The apparatus of claim 11, wherein the apparatus is caused to perform the generation of the estimation of the drying shrinkage of the veneer sheet by scaling a value representing a preliminary drying shrinkage with a value derivable from at least one measurement value representing moisture.

17. The apparatus of claim 11, the apparatus is further caused to generate a control signal to a cutting device in accordance with the estimation of the drying shrinkage of the veneer sheet to cause a cutting of the veneer sheet from the veneer ribbon.

18. A method for generating an estimation of a drying shrinkage of a veneer sheet, the method, performed by an apparatus, comprises:
receiving data from a density measurement device the data received from the density measurement device representing a density of the veneer sheet; and
receiving data from at least one other entity, the data received from the at least one other entity representing at least one characteristic of the veneer sheet,
generating an estimation of a drying shrinkage of the veneer sheet based on the received data.

19. The method of claim 18, wherein the method comprises applying a classification function to the data representing at least one characteristic of the veneer sheet for classifying the veneer sheet to a class among a plurality of classes in accordance with the data representing at least one characteristic of the veneer sheet.

20. The method of claim 19, wherein the method one or more internal parameters defined individually for each of the classes are accessed to, wherein the one or more internal parameters are combined with the data received from the density measurement device for generating the estimation of the drying shrinkage of the veneer sheet.

21. The method of claim 20, wherein the method the one or more internal parameters and the data received from the density measurement device are combined by using internal parameters of a class as constants of formulas and using respective values representing density characteristics of the veneer sheet as parameters of respective formulas.

22. The method of claim 18, wherein an estimation model for estimating the drying shrinkage is selected among a plurality of estimation models in accordance with a value representing of the moisture of the veneer sheet.

23. The method of claim 18, wherein the generation of the estimation of the drying shrinkage of the veneer sheet is performed by scaling a value representing a preliminary drying shrinkage with a value derivable from at least one measurement value representing moisture.

24. The method of claim 18, the method further comprising: generating a control signal to a cutting device in accordance with the estimation of the drying shrinkage of the veneer sheet to cause a cutting of the veneer sheet from the veneer ribbon.

25. A non-transitory computer-readable medium on which is stored a computer program product for generating an estimation of a drying shrinkage of a veneer sheet, which computer program product, when executed by at least one processor, cause an apparatus to perform the method according to claim 18.
